(19) United States
(12) Patent Application Publication
(10) Pub. No.: US 2022/0323430 A1
(43) Pub. Date: Oct. 13, 2022

(54) METHODS OF MANUFACTURING ANAMORELIN TABLETS HAVING IMPROVED STABILITY

(71) Applicant: HELSINN HEALTHCARE SA, Lugano/Pazzallo (CH)

(72) Inventors: Koji Taniguchi, Osaka (JP); Kaori Iida, Osaka (JP); Asuka Hayashi, Osaka (JP); Eleanor De Groot, Houston, TX (US)

(21) Appl. No.: 17/634,616

(22) PCT Filed: Aug. 28, 2020
(86) PCT No.: PCT/IB2020/058064

§ 371 Date: Feb. 11, 2022

Related U.S. Application Data
(60) Provisional application No. 62/893,822, filed on Aug. 30, 2019.

(51) Int. Cl. A61K 31/454; A61K 9/20

(52) U.S. Cl. CPC A61K 31/454; A61K 9/2009; A61K 9/2018; A61K 9/2054

(57) ABSTRACT

Methods for reducing the formation of impurities in finished dosage forms of anamorelin hydrochloride, including formulations for improving such stability and analytical techniques for controlling impurity formation.

### FIELD OF THE INVENTION

The present disclosure relates to anamorelin hydrochloride, formulations of anamorelin hydrochloride having improved stability, methods of manufacturing such formulations, methods of treatment using such formulations, and methods of reducing and controlling for impurity formation.

### BACKGROUND OF THE INVENTION

Anamorelin is a synthetic orally active compound originally synthesized in the 1990s as a growth hormone secretagogue currently under development for the treatment of cancer related cachexia. The free base of anamorelin is chemically defined as:
(3R)1-(2-methylalanyl-D-tryptophyl)-3-(phenylmethyl)-3-piperidinecarboxylic acid 1,2,2trimethylhydrazide,
3-{(2R)-3-{(3R)-3-benzyl-3-[(trimethylhydrazino)carbonyl]piperidin-1-yl}-2-[(2-methylalanyl)amino]-3-oxopropyl}-1H-indole, or
2-Amino-N-[(1R)-2-[(3R)-3-benzyl-3-(N,N′,N′-trimethylhydrazinocarbonyl) piperidin-1-yl]-1-(1H-indol-3-ylmethyl)-2-oxoethyl]-2-methylpropionamide, and has the below chemical structure:

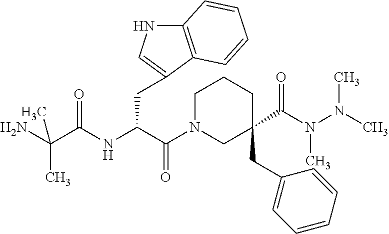 [SMILES: CN(C)N(C)C(=O)[C@@]1(Cc2ccccc2)CCCN(C(=O)[C@@H](Cc2c[nH]c3ccccc23)NC(=O)C(C)(C)N)C1]

A commercial dosage form is being developed as the hydrochloride salt by Ono Pharmaceuticals (Osaka Japan) and Helsinn Healthcare (Lugano Switzerland).

WO 01/34593 of Ankersen et al. describes a method of preparing anamorelin as the fumarate salt, with the hydrochloride salt produced as an intermediate in Step (j) of Example 1. WO 2006/016995 of Lorimer et al. describes a process for preparing crystal forms of the free base of anamorelin. WO 2013/158874 of Kuwabe et al. describes a method of producing anamorelin hydrochloride with controlled chloride content and low residual solvents. WO 2016/036598 of Mann et al. describes methods of using anamorelin hydrochloride for the treatment of cancer cachexia. Other methods of using anamorelin are described in WO 2010/099522 of Polvino et al. and WO 2008/100448 of Polvino et al.

In spite of the foregoing developments, methods for preventing the formation of unwanted degradants of anamorelin hydrochloride are needed, particularly when anamorelin hydrochloride having excess of chloride is formulated into pharmaceutically acceptable dosage forms. Controlling the formation of anamorelin impurity A, an analog and degradant of anamorelin hydrochloride having an HPLC response factor of 1.53 relative to anamorelin, has become especially important.

### SUMMARY OF INVENTION

It has unexpectedly been discovered that certain tableting excipients improve the stability of anamorelin hydrochloride when compressed with anamorelin hydrochloride into tablets, and that these excipients prevent the degradation of anamorelin hydrochloride to impurity A. Without wishing to be bound by any theory, it is believed that these excipients, when intimately admixed with the anamorelin hydrochloride, either physically or chemically sequester the hydrochloride from the anamorelin free base molecule and thereby prevent the degradation of anamorelin to impurity A.

Therefore, in a first principal embodiment the invention provides a method of manufacturing an anamorelin hydrochloride tablet, and tablets made thereby, comprising: (a) admixing anamorelin hydrochloride and one or a combination of pharmaceutically acceptable carriers selected from microcrystalline cellulose, croscarmellose sodium, silicon dioxide, magnesium stearate and anhydrous dibasic calcium phosphate to form an admixture; and (b) compressing said admixture into a tablet.

Other embodiments relate to the discovery of Impurity A, and to methods of manufacturing anamorelin hydrochloride dosage forms by controlling for Impurity A. Thus, in a second principal embodiment, the invention provides a method of manufacturing anamorelin hydrochloride tablets, and tablets made thereby, comprising: (a) admixing anamorelin hydrochloride and a pharmaceutically acceptable carrier means for preventing the formation of impurity A to form an admixture; (b) compressing said admixture into tablets; (c) isolating impurity A from anamorelin hydrochloride in one or more of said tablets; (d) quantifying the amount of impurity A in said one or more tablets; and (e) optionally repeating steps (c) and (d) six months or one year after step (b).

Other embodiments relate to Impurity A itself. Thus, in a third principal embodiment the invention provides impurity A isolated from anamorelin hydrochloride.

Still further embodiments relate to anamorelin hydrochloride tablets themselves. Thus, in a fourth principal embodiment, the invention provides a tablet comprising anamorelin hydrochloride as an active ingredient, which further comprises a pharmaceutically acceptable carrier selected from microcrystalline cellulose, croscarmellose sodium, silicon dioxide, magnesium stearate and anhydrous dibasic calcium phosphate.

In a fifth principal embodiment the invention provides a tablet comprising anamorelin hydrochloride as an active ingredient and pharmaceutically acceptable carrier means for preventing the formation of impurity A.

Still further embodiments relate to the use of anamorelin hydrochloride to treat cancer cachexia using the tablets of the current invention. Thus, in a sixth principal embodiment the invention provides a method for improving one or more symptoms of cancer cachexia in a patient in need thereof comprising administering to said patient a therapeutically effective amount of anamorelin hydrochloride in a tablet according to the present invention, wherein: (a) said patient is characterized by a body mass index less than 25, a score on the Cancer Fatigue Scale of from 20 to 28, or a Quality-of-Life Questionnaire for Cancer Patients Treated With Anticancer Drugs (QOL-ACD) score of from 65 to 80; and (b) said symptoms are selected from the group consisting of lean body mass, appetite, body weight, fatigue, and quality of life.

Additional advantages of the invention are set forth in part in the description which follows, and in part will be obvious from the description or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

### Definitions and Use of Terms

As used in this specification and in the claims which follow, the following terms have the following meanings and use:

The singular forms “a,” “an” and “the” include plural referents unless the context clearly dictates otherwise.

The word “comprise” and variations of the word, such as “comprising” and “comprises,” means “including but not limited to,” and is not intended to exclude, for example, other additives, components, integers or steps. When an element is described as comprising a plurality components, steps or conditions, it will be understood that the element can also be described as comprising any combination of such plurality, or “consisting of” or “consisting essentially of” the plurality or combination of components, steps or conditions.

When testing methods are made by reference to a standard setting organization such as the International Conference on Harmonization (“ICH”), or a testing methodology such as the Cancer Fatigue Scale, it will be understood that the methods are performed according to methods in force as of the earliest priority date of the relevant subject matter. When pharmaceutical testing is required herein it will be understood that the testing is performed in accordance with ICH guidance documents in force as of the earliest priority date of the relevant subject matter, United States Pharmacopoeia (USP) methods in force as of the earliest priority date of the relevant subject matter, or American Society of Testing and Materials (ASTM) methods in force as of the earliest priority date of the relevant subject matter.

The “Cancer Fatigue Scale” refers to the clinical outcome assessment described by Toru Okuyama et al. in Development and Validation of the Cancer Fatigue Scale: A Brief, Three-Dimensional, Self-Rating Scale for Assessment of Fatigue in Cancer Patients. Vol. 19 No. 1 Jan. 2000 Journal of Pain and Symptom Management.

The “Quality of Life Questionnaire for Cancer Patients Treated with Anticancer Drugs” or “QOL-ACD” refers to the clinical outcome assessment published by T. Matsumoto et al. in The quality of life questionnaire for cancer patients treated with anticancer drugs (QOL-ACD): validity and reliability in Japanese patients with advanced non-small-cell lung cancer. Quality of Life Research: an International Journal of Quality of Life Aspects of Treatment, Care and Rehabilitation, 31 Jul. 2002, 11(5):483-493.

When ranges are given by specifying the lower end of a range separately from the upper end of the range, or specifying particular numerical values, it will be understood that a range can be defined by selectively combining any of the lower end variables, upper end variables, and particular numerical values that is mathematically possible. In like manner, when a range is defined as spanning from one endpoint to another, the range will be understood also to encompass a span between and excluding the two endpoints.

When used herein the term “about” will compensate for variability allowed for in the pharmaceutical industry and inherent in products in this industry, such as differences in product strength due to manufacturing variation and time-induced product degradation.

“Anamorelin hydrochloride” refers to a salt of anamorelin and hydrochloric acid in a ratio of approximately 1:1, corresponding to 6.08% of the chloride. The chloride content is preferably less than 6.3% or 6.2% of the molecule, and preferably ranges from 5.7 to 6.3% or from 5.8 to 6.2%. Alternatively, there might be a slight molar excess of chloride, in which case the chloride content might range from 6.1% to 6.3% or 6.1% to 6.2%. Anamorelin hydrochloride defined by any of these ranges can be used in the methods and formulations of the present invention.

“Impurity A” refers to a degradant/analog of anamorelin hydrochloride having an HPLC response factor relative to anamorelin of 1.53 when measured according to the conditions described in Example 3. Alternatively, “Impurity A” has an HPLC relative retention time of 0.34 when the retention time of anamorelin hydrochloride is 1 minute by measuring according to the conditions described in Example 3.

“Pharmaceutically acceptable carrier means for preventing the formation of impurity A” corresponds to the combination of microcrystalline cellulose, croscarmellose sodium, silicon dioxide, magnesium stearate, and anhydrous dibasic calcium phosphate, present in a prevention effective amount when intimately admixed with anamorelin hydrochloride and compressed into a tablet at a hardness adequate to produce a pharmaceutically acceptable immediate release tablet and to fulfill its recited function. Prevention does not require 100% prevention, but it does require a weight ratio of means:anamorelin hydrochloride of from 0.5:1 to 10:1, or any of the more specific ratios described herein, capable of achieving stability equivalent to the stability reported in the examples hereto for such ratios. “Prevention effective amount” means that amount which, when combined with anamorelin hydrochloride in an intimate admixture, and compressed into a tablet, is sufficient to reduce the degradation rate of anamorelin hydrochloride, especially to impurity A. In preferred embodiments, the prevention effective amount is sufficient to prevent the formation of more than about 0.1% or 0.05% impurity A based on the weight of said anamorelin hydrochloride after storage for 6 months at 40° C. and a relative humidity of 75%.

Alternatively, the “Pharmaceutically acceptable carrier means for preventing the formation of impurity A” can be expressed as “Pharmaceutically acceptable carrier means for preventing a 200% increase in the formation of impurity A after storage for 6 months at 40° C. and a relative humidity of 75%,” or as “Pharmaceutically acceptable carrier means for preventing a 100% increase in the formation of impurity A after storage for 6 months at 40° C. and a relative humidity of 75%,” in which case the means will correspond to the formulation capable of producing such a result.

The terms “excipient” and “carrier” are used synonymously herein.

### DISCUSSION

The invention can be defined based on several principal embodiments which can be further defined or modified based on the discussion herein to create additional embodiments. In a first principal embodiment the invention provides a method of manufacturing an anamorelin hydrochloride tablet, and tablets made thereby, comprising: (a) admixing anamorelin hydrochloride and one or a combination of pharmaceutically acceptable carriers selected from microcrystalline cellulose, croscarmellose sodium, silicon dioxide, magnesium stearate and anhydrous dibasic calcium phosphate to form an admixture; and (b) compressing said admixture into a tablet.

In a second principal embodiment, the invention provides a method of manufacturing anamorelin hydrochloride tablets, and tablets made thereby, comprising: (a) admixing anamorelin hydrochloride and a pharmaceutically acceptable carrier means for preventing the formation of impurity A to form an admixture; (b) compressing said admixture into tablets; (c) isolating impurity A from anamorelin hydrochloride in one or more of said tablets; (d) quantifying the amount of impurity A in said one or more tablets; and (e) optionally repeating steps (c) and (d) six months or one year after step (b).

In a third principal embodiment the invention provides impurity A isolated from anamorelin hydrochloride.

In a fourth principal embodiment, the invention provides a tablet comprising anamorelin hydrochloride as an active ingredient, which further comprises a pharmaceutically acceptable carrier selected from microcrystalline cellulose, croscarmellose sodium, silicon dioxide, magnesium stearate and anhydrous dibasic calcium phosphate.

In a fifth principal embodiment the invention provides a tablet comprising anamorelin hydrochloride as an active ingredient and pharmaceutically acceptable carrier means for preventing the formation of impurity A.

In a sixth principal embodiment the invention provides a method for improving one or more symptoms of cancer cachexia in a patient in need thereof comprising administering to said patient a therapeutically effective amount of anamorelin hydrochloride in a tablet according to the present invention, wherein: (a) said patient is characterized by a body mass index less than 25, a score on the Cancer Fatigue Scale of from 20 to 28, or a Quality-of-Life Questionnaire for Cancer Patients Treated With Anticancer Drugs (QOL-ACD) score of from 65 to 80; and (b) said symptoms are selected from the group consisting of lean body mass, appetite, body weight, fatigue, and quality of life.

### Tablet Characteristics

Preferred carriers that have been found to improve the stability of anamorelin hydrochloride when compressed with anamorelin hydrochloride into a tablet are selected from microcrystalline cellulose, croscarmellose sodium, silicon dioxide, magnesium stearate, anhydrous dibasic calcium phosphate, lactose monohydrate, D-mannitol, corn starch, low-substituted hydroxypropylcellulose, sodium starch glycolate, carmellose calcium, carmellose, crospovidone, partially pregelatinized maize starch, stearic acid and sodium stearyl fumarate. A tablet can be formulated and manufactured, to produce a pharmaceutically acceptable and pharmaceutically stable product, based on the teachings of this invention and common general knowledge of pharmaceutical formulation techniques.

The tablet can comprise only one of these preferred carriers or any combination of these preferred carriers. Thus, in one subembodiment the tablet comprises two or more of these preferred carriers. In another subembodiment the tablet comprises three or more of these preferred carriers. In another subembodiment the tablet comprises four or more of these preferred carriers.

However based on the examples provided in this document, one might choose to avoid the use of mannitol and HPC or their pharmaceutical equivalents. Thus, in one embodiment the formulations of the present invention omit sugar alcohols such as mannitol. In another embodiment the formulations of the present invention omit mannitol, sorbitol, and/or xylitol. In still another embodiment the formulations of the present invention omit mannitol. In other embodiments, the formulations of the present invention omit HPC and/or HPMC.

The tablet will preferably comprise one or a combination of the preferred carriers in an amount sufficient to prevent the degradation of anamorelin hydrochloride into impurity A during storage (a “prevention effective amount”). This “prevention effective amount” can further be described in terms of the quantity of a preferred excipient or combination of preferred excipients in the formulation relative to the anamorelin hydrochloride. Thus, any of the preferred excipients can be used in an amount of from about 0.01 to about 20 parts by weight based on 1 part by weight of anamorelin hydrochloride. Alternatively, any of the preferred excipients can be used in an amount of from about 0.5 to about 10 parts by weight based on 1 part by weight of anamorelin hydrochloride. As another alternative, any of the preferred excipients can be used in an amount of from about 1 to about 6 parts by weight based on 1 part by weight of anamorelin hydrochloride. As another alternative, any of the preferred excipients can be used in an amount of from about 0.01, 0.1, 1, 5, 10 or 20 parts by weight or more based on 1 part by weight of anamorelin hydrochloride.

The prevention effective amount can also be defined based on the amount of a preferred excipient present in the formulation sufficient to perform its conventional tableting function, as a diluent, disintegrating agent, glidant, or lubricant, in addition to its stabilizing function. Thus, in various aspects, the microcrystalline cellulose, anhydrous dibasic calcium phosphate, lactose monohydrate, D-mannitol or corn starch are present independently (i.e. only one of the carriers is present) or in combination in an amount of from about 1 to about 10 parts by weight relative to one weight part anamorelin hydrochloride. In other aspects the croscarmellose sodium is present in an amount of from about 0.1 to about 2 parts by weight relative to one weight part anamorelin hydrochloride. In other aspects the silicon dioxide is present in an amount of from about 0.01 to about 0.2 weight parts relative to one weight part anamorelin hydrochloride. In other aspects the magnesium stearate is present in an amount of from about 0.01 to about 0.2 weight parts relative to one weight part anamorelin hydrochloride. In other aspects the low-substituted hydroxypropylcellulose is present in an amount of from about 0.01 to about 0.2 weight parts relative to one weight part anamorelin hydrochloride. In other aspects the sodium starch glycolate is present in an amount of from about 0.01 to about 0.2 weight parts relative to one weight part anamorelin hydrochloride. In other aspects the carmellose calcium is present in an amount of from about 0.01 to about 0.2 weight parts relative to one weight part anamorelin hydrochloride. In other aspects the carmellose is present in an amount of from about 0.01 to about 0.2 weight parts relative to one weight part anamorelin hydrochloride. In other aspects the crospovidone is present in an amount of from about 0.01 to about 0.2 weight parts relative to one weight part anamorelin hydrochloride. In other aspects the partially pregelatinized maize starch is present in an amount of from about 0.01 to about 0.2 weight parts relative to one weight part anamorelin hydrochloride. In other aspects the stearic acid is present in an amount of from about 0.01 to about 0.2 weight parts relative to one weight part anamorelin hydrochloride. In other aspects the sodium stearyl fumarate is present in an amount of from about 0.01 to about 0.2 weight parts relative to one weight part anamorelin hydrochloride. It will be understood that any of these preferred excipients can be present by itself or in combination with another preferred excipient at these weight parts.

In other embodiments the prevention effective amount is based on the weight of the entire combination of preferred carriers in the tablet relative to the anamorelin hydrochloride. Thus, in one aspect the sum of the preferred carriers in the tablet is from about 0.01 to about 20 parts by weight based on 1 part by weight of anamorelin hydrochloride. In an alternative aspect the sum of preferred carriers in the tablet is from about 0.5 to about 10 parts by weight based on 1 part by weight of anamorelin hydrochloride. In another alternative aspect the sum of the preferred carriers in the tablet is from about 1 to about 6 parts by weight based on 1 part by weight of anamorelin hydrochloride. Once again, the tablet does not need to include all of the preferred excipients, but those excipients that are present are preferably summed to satisfy the foregoing weight parts.

In another subembodiment the tablet is defined by its stability. Thus, in various subembodiment the tablets of the current invention are defined as tablets in which impurity A is not substantially produced or the amount of impurity A produced after storage for 2 to 6 months at 40° C. and a relative humidity of 75% is less than about 0.3% or 0.05%, preferably 0.1% or 0.05% based on the weight of said anamorelin hydrochloride.

The tablet can further be defined in terms of its hardness. Thus, in any of the embodiments of the present invention the tablet can have a hardness of from about 40 to about 200 Newtons. Alternatively or in addition, in any of the embodiments of the present invention, the pharmaceutically acceptable carrier(s) can be compressed with said anamorelin hydrochloride at a compression force of from about 0.5 to about 15 kN.

In any of the embodiments of this invention, the anamorelin hydrochloride and preferred carriers will be intimately admixed. I.e., they will each be homogeneously dispersed throughout the tablet.

The tablet can be coated or uncoated but, in a preferred embodiment, the tablet is coated using traditional coating excipients.

In yet another aspect, the tablet is characterized by its method of manufacture, and comprises a tablet made by any of the methods described herein.

### Carrier Means

The tablets of the current invention can also be described in terms of the means used to achieve the surprising stability. This means will be referred to herein as “pharmaceutically acceptable carrier means for preventing the formation of impurity A,” or simply “carrier means.” The tablet described in any of the embodiments of the current invention will comprise anamorelin hydrochloride and such carrier means in a prevention effective amount. As mentioned in the Tablet Characteristics and Methods of Manufacture sections of this document, these preferred excipients are most effective when intimately admixed with anamorelin hydrochloride and compressed into a tablet. An exemplary compression force is from about 0.5 to about 15 kN. An exemplary tablet hardness is from about 40 to about 200 Newtons.

Therefore, in one aspect the pharmaceutically acceptable carrier means is in an intimate admixture with said anamorelin hydrochloride and compressed with said anamorelin hydrochloride at a compression force of from about 0.5 to about 15 kN.

In another aspect the pharmaceutically acceptable carrier is in an intimate admixture with said anamorelin hydrochloride and compressed to a hardness of from about 40 to about 200 Newtons.

In yet another aspect the tablet comprises from about 0.01 to about 20, from about 0.5 to about 10, or from about 1 to about 6 parts by weight of said pharmaceutically acceptable carrier means or one or a combination of said pharmaceutically acceptable carriers based on 1 part by weight of anamorelin hydrochloride.

As mentioned previously, it is believed that these preferred excipients prevent the formation of impurity A by chemically or physically sequestering the hydrochloride from the anamorelin moiety. Thus, in one aspect, the pharmaceutically acceptable carrier means act as HCl sequestrants.

### Methods of Manufacture

The disclosed pharmaceutical tablets can be prepared by any of the well-known techniques of pharmacy. Formulation of drugs is discussed in, for example, Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999. However, in a preferred embodiment the tablets are produced according to one of the principal embodiments of the current invention.

In one aspect, an intimate admixture of anamorelin hydrochloride and one or a combination of the preferred carriers discussed herein, preferably at any of the weight ratios discussed in the Tablet Characteristics section of this document, in a prevention effective amount, is compressed into a tablet, preferably at a compression force of from about 0.5 to about 15 kN. In another aspect, an intimate admixture of anamorelin hydrochloride and the carrier means discussed in the Carrier Means section of this document, preferably at the weight ratios discussed in the Tablet Characteristics section of this document, in a prevention effective amount, is compressed into a tablet, preferably at a compression force of from about 0.5 to about 15 kN. Conventional excipients other than the preferred carriers discussed herein can also be employed according to known pharmaceutical manufacturing techniques. The tablet also can be coated by one or more coating excipients according to methods well known in the art.

Thus, in various subembodiments, the methods of manufacture are practiced by admixing anamorelin hydrochloride and two or more, three or more, or four or more of the preferred carriers. In other subembodiments, the pharmaceutically acceptable carrier means comprises two or more, three or more, or four or more of the preferred carriers.

In like manner, the methods of manufacture can be practiced by admixing from about 0.01 to about 20 parts by weight, from about 0.5 to about 10 parts by weight, or from about 1 to about 6 parts by weight, of one or a combination of the preferred carriers based on 1 part by weight of anamorelin hydrochloride. Conversely, the pharmaceutically acceptable carrier means can comprise from about 0.01 to about 20 parts by weight, from about 0.5 to about 10 parts by weight, or from about 1 to about 6 parts by weight, of said one or a combination of the preferred carriers based on 1 part by weight of anamorelin hydrochloride.

In any event, the preferred carriers or pharmaceutically acceptable carrier means is preferably present in an amount sufficient to prevent the formation of impurity A. In terms of the amount of impurity A produced after storage for 6 months at 40° C. and a relative humidity of 75%, suitable percentages range from 0.5% to 0.001%, from 0.2% to 0.001%, and from 0.1% to 0.001%, based on the weight of said anamorelin hydrochloride. However, suitable percentages preferably range from 0.15% to 0.001%, from 0.10% to 0.001%, or from 0.07% to 0.001%. Alternatively, the stability of the dosage form can be measured in terms of the increased amount of impurity A produced after storage for 6 months at 40° C. and a relative humidity of 75%. Thus, in alternative embodiments, the percentage of impurity A produced after storage for 6 months at 40° C. and a relative humidity of 75% is less than 3× the percentage of impurity A at t₀, less than 2× the percentage of impurity A at t₀, or less than 1.5× the percentage of impurity A at t₀.

Once manufactured, impurity A will preferably be isolated from anamorelin hydrochloride, preferably according to the HPLC methods described herein, and the tablet preferably analyzed for impurity A according to the methods described herein. Thus, when impurity A is separated in the methods of the current invention, it is preferably isolated by dissolving one or more tablets in an organic solvent, and separating anamorelin hydrochloride from impurity A by high performance liquid chromatography.

### Methods of Analysis

The unexpected stability and purity of the tablets of the current invention is due in large part to the discovery of impurity A, the isolation of impurity A from anamorelin through HPLC, and methods of measuring the amount of impurity A in a given tablet using HPLC. Thus, in one embodiment the invention provides a method of controlling the formation of impurities in anamorelin tablets by measuring the concentration of impurity A by HPLC. In another embodiment the invention provides impurity A isolated from anamorelin hydrochloride. In one embodiment impurity A is present in a non-polar organic solvent. In another embodiment impurity A is present in a solution comprising water, trifluoroacetic acid, and acetonitrile.

In still further embodiments, the invention provides methods of analyzing for impurity A during the manufacture of anamorelin hydrochloride tablets, and after the manufacture of the tablets in a defined stability program. For example, tablets from a given batch can be analyzed for Impurity A at six months or one year after the batch is manufactured. Impurity A is an analog or degradant of anamorelin hydrochloride that has a response factor of 1.53 relative to anamorelin hydrochloride during high performance liquid chromatography. The conditions in which impurity A displays a response factor of 1.53 during HPLC are detailed more specifically in example 3 in this document.

### Methods of Treatment

As previously mentioned, the invention further comprises methods of treatment using the tablets of the current invention. In various subembodiments, lean body mass is estimated by dual-energy x-ray absorptiometry (DEXA), the fatigue is measured by the Cancer Fatigue Scale, and the quality of life is measured by a QOL-ACD score for items 7 to 11 (“physical condition”), item 8 (“Did you have a good appetite?”), item 9 (“Did you enjoy your meals?”), and item 11 (“Did you lose any weight?”). In other subembodiments, the patient has stage III or IV non-small cell lung cancer (NSCLC) or advanced gastrointestinal (colorectal, gastric, or pancreatic) cancer.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### Example 1. Evaluation of Stability of Tablets Containing Anamorelin Hydrochloride and a Single Pharmaceutically Acceptable Carrier

Anamorelin hydrochloride (Lot A and Lot B) was mixed with different excipients at a weight ratio of 1:10 or 1:1 (anamorelin hydrochloride:excipient) and compressed into tablets containing 50 mg of anamorelin hydrochloride as reported in Table 1 and Table 2.

TABLE 1

| | Compar- | | | | | |
|---|---|---|---|---|---|---|
| | ative | | | | | |
| | Formu- | | | | | |
| | lation | Form. | Form. | Form. | Form. | Form. |
| | (Lot A) | 1 | 2 | 3 | 4 | 5 |
| anamorelin | 50 mg | 50 mg | 50 mg | 50 mg | 50 mg | 50 mg |
| hydrochloride | | | | | | |
| (Lot A) | | | | | | |
| microcrystalline | — | 500 mg | — | — | — | — |
| cellulose | | | | | | |
| anhydrous | — | — | 500 mg | — | — | — |
| dibasic | | | | | | |
| calcium | | | | | | |
| phosphate | | | | | | |
| croscarmellose | — | — | — | 50 mg | — | — |
| sodium | | | | | | |
| silicon dioxide | — | — | — | — | 50 mg | — |
| magnesium | — | — | — | — | — | 50 mg |
| stearate | | | | | | |
| total | 50 mg | 550 mg | 550 mg | 100 mg | 100 mg | 100 mg |

TABLE 2

| | Comparative | | | | | | | | | |
|---|---|---|---|---|---|---|---|---|---|---|
| | Formulation | | | | | | | | | |
| | (Lot B) | Form. a | Form. b | Form. c | Form. d | Form. e | Form. f | Form. g | Form. h | Form. i |
| anamorelin | 50 mg | 50 mg | 50 mg | 50 mg | 50 mg | 50 mg | 50 mg | 50 mg | 50 mg | 50 mg |
| hydrochloride | | | | | | | | | | |
| (Lot B) | | | | | | | | | | |
| lactose | — | 500 mg | — | — | — | — | — | — | — | — |
| monohydrate | | | | | | | | | | |
| D-mannitol | — | — | 500 mg | — | — | — | — | — | — | — |
| corn starch | — | — | — | 500 mg | — | — | — | — | — | — |
| low-substituted | — | — | — | — | 50 mg | — | — | — | — | — |
| hydroxypropyl- | | | | | | | | | | |
| cellulose | | | | | | | | | | |
| sodium starch | — | — | — | — | — | 50 mg | — | — | — | — |
| glycolate | | | | | | | | | | |
| carmellose | — | — | — | — | — | — | 50 mg | — | — | — |
| calcium | | | | | | | | | | |
| carmellose | — | — | — | — | — | — | — | 50 mg | — | — |
| crospovidone | — | — | — | — | — | — | — | — | 50 mg | — |
| partially | — | — | — | — | — | — | — | — | — | 50 mg |
| pregelatinized | | | | | | | | | | |
| maize starch | | | | | | | | | | |
| total | 50 mg | 550 mg | 550 mg | 550 mg | 100 mg | 100 mg | 100 mg | 100 mg | 100 mg | 100 mg |

The stability of these tablets was measured after two months of storage under accelerated study conditions of temperature and relative humidity as described in ICH Q1A (R2), in a closed bottle, and compared to the stability of tablets containing 100% anamorelin monohydrochloride (50 mg). Stability was determined by measuring the content of impurity A under the HPLC conditions reported in Example 3. The results of the stability testing are reported in Table 3 and Table 4.

TABLE 3

| Storage condition | | 40° C./75% RH | | | |
|---|---|---|---|---|---|
| Storage duration | | Initial | 1 month | 2 months | 6 months |
| Concentration | Comparative | 0.02 | 0.04 | 0.07 | 0.06 |
| of impurity A | Formulation | | | | |
| in anamorelin | (Lot A) | | | | |
| hydrochloride | Form. 1 | 0.02 | 0.02 | 0.02 | 0.02 |
| 50 mg | Form. 2 | 0.02 | 0.02 | 0.02 | 0.03 |
| | Form. 3 | 0.02 | N.T. | 0.03 | 0.03 |
| | Form. 4 | 0.02 | 0.04 | 0.04 | 0.04 |
| | Form. 5 | 0.02 | 0.02 | 0.03 | 0.03 |

TABLE 4

| | | Storage condition | | | | |
|---|---|---|---|---|---|---|
| | | 40° C./75% RH | | | | |
| | | Storage duration | | | | |
| | | | 1 | 2 | 3 | 6 |
| | | Initial | month | months | months | months |
| Concentration of | Comparative | 0.04 | 0.18 | 0.25 | 0.37 | 0.52 |
| impurity A in | Formulation | | | | | |
| anamorelin | (Lot B) | | | | | |
| hydrochloride | Form. a | 0.06 | 0.07 | 0.08 | 0.08 | 0.08 |
| 50 mg | Form. b | 0.04 | 0.10 | 0.17 | 0.12 | 0.13 |
| | Form. c | 0.04 | 0.05 | 0.07 | 0.04 | 0.07 |
| | Form. d | 0.03 | 0.11 | 0.12 | 0.14 | 0.21 |
| | Form. e | 0.03 | 0.04 | 0.05 | 0.04 | 0.04 |
| | Form. f | 0.03 | 0.05 | 0.06 | 0.04 | 0.05 |
| | Form. g | 0.03 | 0.06 | 0.08 | 0.09 | 0.08 |
| | Form. h | 0.03 | 0.11 | 0.05 | 0.06 | 0.05 |
| | Form. i | 0.03 | 0.06 | 0.08 | 0.07 | 0.07 |

### Example 2: Evaluation of Stability of Tablets Containing Anamorelin Hydrochloride and a Combination of Pharmaceutically Acceptable Carriers

In the same experiment as Example 1, a combination of pharmaceutically acceptable carriers was mixed with anamorelin hydrochloride at three different weight ratios (1:1, 1:3 and 1:6) or (1:1, 3:2 and 3:1) (anamorelin:excipient mixture) and the mixture compressed to make tablets containing 50 mg or 150 mg anamorelin hydrochloride as reported in Table 5, Table 6 and Table 7.

TABLE 5

| | Form. 6 | Form. 7 | Form. 8 | Form. 9 | Form. 10 | Form. 11 |
|---|---|---|---|---|---|---|
| anamorelin hydrochloride | 50 mg | 50 mg | 50 mg | | | |
| (Lot A) | | | | | | |
| anamorelin hydrochloride | | | | 50 mg | 75 mg | 75 mg |
| (Lot B) | | | | | | |
| mixture of microcrystalline | 50 mg | 150 mg | 300 mg | 50 mg | 50 mg | 25 mg |
| cellulose, croscarmellose | | | | | | |
| sodium, silicon dioxide and | | | | | | |
| magnesium stearate | | | | | | |
| total | 100 mg | 200 mg | 350 mg | 100 mg | 125 mg | 100 mg |

TABLE 6

| | Form. j | Form. k | Form. l |
|---|---|---|---|
| anamorelin hydrochloride (Lot B) | 50 mg | 75 mg | 75 mg |
| mixture of lactose monohydrate, | 50 mg | 50 mg | 25 mg |
| corn starch, sodium starch | | | |
| glycolate and stearic acid | | | |
| total | 100 mg | 125 mg | 100 mg |

TABLE 7

| | Form. m | Form. n | Form. o |
|---|---|---|---|
| anamorelin hydrochloride (Lot B) | 50 mg | 75 mg | 75 mg |
| mixture of D-mannitol, | 50 mg | 50 mg | 25 mg |
| corn starch, low-substituted | | | |
| hydroxypropylcellulose | | | |
| and sodium stearyl fumarate | | | |
| total | 100 mg | 125 mg | 100 mg |

The stability of these tablets was measured after two months of storage under accelerated study conditions of temperature and relative humidity as described in ICH QA (R2), in a closed bottle and compared to the stability of the comparative formulation described in Example 1. Stability was determined by measuring the concentration of impurity A under the HPLC conditions reported in Example 3. The results of the stability testing are reported in Table 8, Table 9, Table 10 and Table 11.

TABLE 8

| Storage condition | | 40° C./75% RH | | | |
|---|---|---|---|---|---|
| Storage duration | | Initial | 1 month | 2 months | 6 months |
| Concentration | Comparative | 0.02 | 0.04 | 0.07 | 0.06 |
| of impurity A | Formulation | | | | |
| in anamorelin | (Lot A) | | | | |
| hydrochloride | Form. 6 | 0.02 | 0.03 | 0.03 | 0.03 |
| 50 mg | Form. 7 | 0.02 | 0.02 | 0.02 | 0.02 |
| | Form. 8 | 0.02 | 0.00 | 0.02 | 0.02 |

TABLE 9

| | | Storage condition | | | | |
|---|---|---|---|---|---|---|
| | | 40° C./75% RH | | | | |
| | | Storage duration | | | | |
| | | | 1 | 2 | 3 | 6 |
| | | Initial | month | months | months | months |
| Concentra- | Comparative | 0.04 | 0.18 | 0.25 | 0.37 | 0.52 |
| tion | Formulation | | | | | |
| of impurity | (Lot B) | | | | | |
| A in | Form. 9 | 0.03 | 0.07 | 0.07 | 0.07 | 0.07 |
| anamorelin | (not- | | | | | |
| hydro- | compressed) | | | | | |
| chloride | Form. 9 | 0.03 | 0.06 | 0.04 | 0.04 | 0.04 |
| | Form. 10 | 0.03 | 0.05 | 0.06 | 0.04 | 0.05 |
| | Form. 11 | 0.00 | 0.06 | 0.07 | 0.08 | 0.09 |

TABLE 10

| | | Storage condition | | | | |
|---|---|---|---|---|---|---|
| | | 40° C./75% RH | | | | |
| | | Storage duration | | | | |
| | | | 1 | 2 | 3 | 6 |
| | | Initial | month | months | months | months |
| Concentration of | Comparative | 0.04 | 0.18 | 0.25 | 0.37 | 0.52 |
| impurity A | Formulation | | | | | |
| in anamorelin | (Lot B) | | | | | |
| hydrochloride | Form. j | 0.03 | 0.06 | 0.04 | 0.07 | 0.07 |
| | (not- | | | | | |
| | compressed) | | | | | |
| | Form. j | 0.03 | 0.05 | 0.04 | 0.04 | 0.04 |
| | Form. k | 0.03 | 0.05 | 0.06 | 0.06 | 0.08 |
| | Form. l | 0.04 | 0.10 | 0.15 | 0.19 | 0.27 |

TABLE 11

| | | Storage condition | | | | |
|---|---|---|---|---|---|---|
| | | 40° C./75% RH | | | | |
| | | Storage duration | | | | |
| | | | 1 | 2 | 3 | 6 |
| | | Initial | month | months | months | months |
| Concentration of | Comparative | 0.04 | 0.18 | 0.25 | 0.37 | 0.52 |
| impurity A | Formulation | | | | | |
| in anamorelin | (Lot B) | | | | | |
| hydrochloride | Form. m | 0.03 | 0.06 | 0.04 | 0.07 | 0.07 |
| | (not- | | | | | |
| | compressed) | | | | | |
| | Form.m | 0.03 | 0.04 | 0.04 | 0.09 | 0.04 |
| | Form. n | 0.03 | 0.07 | 0.07 | 0.08 | 0.11 |
| | Form. o | 0.04 | 0.10 | 0.17 | 0.22 | 0.27 |

### Example 3: HPLC Method of Analyzing for Impurity A

Each sample was dissolved in the following mobile phase A/mobile phase B mixture (17:3) to make each test sample. 10 μg of each test sample was then tested by HPLC in the conditions described in Table 5. Peak area (At) of test samples was measured by automated integration. The concentrations of anamorelin hydrochloride and impurity A were calculated by the following formula.

Concentration of analogue (%)=At/Aa×RRF×100
At: Each peak area of test sample
Aa: The total of all peak area
RRF: Relative reaction factor (impurity A: 1.53)

TABLE 12

| Detector | UV 280 nm | | |
|---|---|---|---|
| Column | Zorbax Bonus RP* | | |
| | (4.6 mm × 250 mm, 3.5 μm, Agilent) | | |
| Column | 55° C. | | |
| temperature | | | |
| Mobile phase | Mobile phase A: 0.1% | | |
| | Trifluoroacetic acid aqueous solution | | |
| | Mobile phase B: 0.1% | | |
| | Trifluoroacetic acid acetonitrile solution | | |
| | Gradient | | |
| | Time (min.) | Phase A (%) | Phase B (%) |
| | 0 | 84 | 16 |
| | 12 | 74 | 26 |
| | 26 | 69.5 | 30.5 |
| | 29 | 69.5 | 30.5 |
| | 41 | 64 | 36 |
| | 50 | 7 | 93 |
| | 54 | 7 | 93 |

| | |
|---|---|
| Flow | 0.85 mL/min |
| Run duration | 54 min |
| Injection volume | 10 μL |

*See Agilent Zorbax Bonus RP Product Datasheet (Aug. 30, 2003)

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A method of manufacturing an anamorelin hydrochloride tablet, comprising:
a) admixing anamorelin hydrochloride and one or a combination of pharmaceutically acceptable carriers selected from microcrystalline cellulose, croscarmellose sodium, silicon dioxide, magnesium stearate, anhydrous dibasic calcium phosphate, lactose monohydrate, D-mannitol, corn starch, low-substituted hydroxypropylcellulose, sodium starch glycolate, carmellose calcium, carmellose, crospovidone, partially pregelatinized maize starch, stearic acid, and sodium stearyl fumarate to form an admixture; and
b) compressing said admixture into a tablet.

2) The method according to claim 1, comprising admixing anamorelin hydrochloride and two or more of said pharmaceutically acceptable carriers.

3) The method according to claim 2, comprising admixing anamorelin hydrochloride and three or more of said pharmaceutically acceptable carriers.

4) The method according to claim 3, comprising admixing anamorelin hydrochloride and four or more of said pharmaceutically acceptable carriers.

5) The method according to any one of claims 1 to 4, comprising admixing from about 0.01 to about 20 parts by weight of said one or a combination of pharmaceutically acceptable carriers based on 1 part by weight of anamorelin hydrochloride.

6) The method according to claim 5, comprising admixing from about 0.5 to about 10 parts by weight of said one or a combination of pharmaceutically acceptable carriers based on 1 part by weight of anamorelin hydrochloride.

7) The method according to claim 6, comprising admixing from about 1 to about 6 parts by weight of said one or a combination of pharmaceutically acceptable carriers based on 1 part by weight of anamorelin hydrochloride.

8) The method according to any one of claims 1 to 7, wherein said admixture is compressed into a tablet at a compression force of from about 0.5 to about 15 kN.

9) The method according to any of claims 1 to 8, wherein said admixture is compressed to a hardness of from about 40 to about 200 Newtons.

10) The method according to any one of claims 1 to 9, wherein said one or a combination of said pharmaceutically acceptable carriers is present in an amount sufficient to prevent the formation of impurity A.

11) The method according to claim 10, wherein the amount of impurity A produced after storage for 2 to 6 months at 40° C. and a relative humidity of 75% is less than about 0.1% based on the weight of said anamorelin hydrochloride.

12) The method according to claim 11, wherein the amount of impurity A produced after storage for 2 months at 40° C. and a relative humidity of 75% is less than about 0.05% based on the weight of said anamorelin hydrochloride.

13) The method according to any one of claims 10 to 12, wherein impurity A has a response factor of 1.53 relative to anamorelin hydrochloride during high performance liquid chromatography.

14) The method according to any one of claims 10 to 12, wherein impurity A has a response factor of 1.53 relative to anamorelin hydrochloride during high performance liquid chromatography as described in Example 3.

15) A method of manufacturing anamorelin hydrochloride tablets, comprising:
a) admixing anamorelin hydrochloride and a pharmaceutically acceptable carrier means for preventing the formation of impurity A to form an admixture;
b) compressing said admixture into tablets;
c) isolating impurity A from anamorelin hydrochloride in one or more of said tablets;
d) quantifying the amount of impurity A in said one or more tablets; and
e) optionally repeating steps (c) and (d) six months or one year after step (b).

16) The method according to claim 15, wherein said isolating step (c) comprises dissolving one or more of said tablets in an organic solvent, and separating said anamorelin hydrochloride from said impurity A by high performance liquid chromatography.

17) The method of any of claims 15 and 16, wherein said pharmaceutically acceptable carrier means act as HCl sequestrants in an intimate admixture with said anamorelin hydrochloride.

18) The method according to any one of claims 15 and 16, wherein said carrier means comprises a prevention effective amount for preventing the formation of impurity A of one or a combination of carriers selected from microcrystalline cellulose, croscarmellose sodium, silicon dioxide, magnesium stearate, anhydrous dibasic calcium phosphate, lactose monohydrate, D-mannitol, corn starch, low-substituted hydroxypropylcellulose, sodium starch glycolate, carmellose calcium, carmellose, crospovidone, partially pregelatinized maize starch, stearic acid and sodium stearyl fumarate.

19) The method according to claim 18, wherein said carrier means comprises two or more of said pharmaceutically acceptable carriers.

20) The method according to claim 19, wherein said carrier means comprises three or more of said pharmaceutically acceptable carriers.

21) The method according to claim 20, wherein said carrier means comprises four or more of said pharmaceutically acceptable carriers.

22) The method according to any one of claims 15 to 20, comprising admixing from about 0.01 to about 20 parts by weight of said carrier means to 1 part by weight of anamorelin hydrochloride.

23) The method according to claim 22, comprising admixing from about 0.5 to about 10 parts by weight of said carrier means to 1 part by weight of anamorelin hydrochloride.

24) The method according to claim 23, comprising admixing from about 1 to about 6 parts by weight of said carrier means to 1 part by weight of anamorelin hydrochloride.

25) The method according to any one of claims 15 to 24, wherein said admixture is compressed into a tablet at a compression force of from about 0.5 to about 15 kN.

26) The method according to any one of claims 15 to 25, wherein said admixture is compressed to a hardness of from about 40 to about 200 Newtons.

27) The method according to any one of claims 15 to 26, wherein said one or a combination of said pharmaceutically acceptable carriers is present in an amount sufficient to prevent the formation of impurity A.

28) The method according to claim 27, wherein the amount of impurity A produced after storage for 2 to 6 months at 40° C. and a relative humidity of 75% is less than about 0.1% based on the weight of said anamorelin hydrochloride.

29) The method according to claim 28, wherein the amount of impurity A produced after storage for 2 months at 40° C. and a relative humidity of 75% is less than about 0.05% based on the weight of said anamorelin hydrochloride.

30) Impurity A isolated from anamorelin hydrochloride.

31) The impurity A according to claim 30 having a response factor of 1.53 relative to anamorelin hydrochloride during high performance liquid chromatography.

32) The impurity A according to claim 31 having a response factor of 1.53 relative to anamorelin hydrochloride during high performance liquid chromatography, as measured in Example 3.

33) The impurity A according to any of claims 30 to 32 in a non-polar organic solvent.

34) The impurity A according to any of claims 30 to 32 in a solution comprising water, trifluoroacetic acid, and acetonitrile.

35) An anamorelin hydrochloride tablet made according to the method of any one of claims 1 to 29.

36) A tablet comprising anamorelin hydrochloride as an active ingredient, which further comprises a pharmaceutically acceptable carrier selected from microcrystalline cellulose, croscarmellose sodium, silicon dioxide, magnesium stearate, anhydrous dibasic calcium phosphate, lactose monohydrate, D-mannitol, corn starch, low-substituted hydroxypropylcellulose, sodium starch glycolate, carmellose calcium, carmellose, crospovidone, partially pregelatinized maize starch, stearic acid and sodium stearyl fumarate.

37) The tablet according to claim 36, comprising two or more of said pharmaceutically acceptable carriers.

38) The tablet according to claim 37, comprising three or more of said pharmaceutically acceptable carriers.

39) The tablet according to claim 38, comprising four or more of said pharmaceutically acceptable carriers.

40) The tablet according to any one of claims 36 to 39, comprising from about 0.01 to about 20 parts by weight of one or a combination of said pharmaceutically acceptable carriers based on 1 part by weight of anamorelin hydrochloride.

41) The tablet according to claim 40, comprising from about 0.5 to about 10 parts by weight of one or a combination of said pharmaceutically acceptable carriers based on 1 part by weight of anamorelin hydrochloride.

42) The tablet according to claim 41, comprising from about 1 to about 6 parts by weight of one or a combination of said pharmaceutically acceptable carriers based on 1 part by weight of anamorelin hydrochloride.

43) The tablet according to any one of claims 36 to 42, wherein impurity A is not substantially produced or the amount of impurity A produced after storage for 2 to 6 months at 40° C. and a relative humidity of 75% is less than about 0.1% based on the weight of said anamorelin hydrochloride.

44) The tablet according to claim 43, wherein impurity A is not substantially produced or the amount of impurity A produced after storage for 2 months at 40° C. and a relative humidity of 75% is less than about 0.05% based on the weight of said anamorelin hydrochloride.

45) The tablet according to claim 43 or 44, wherein impurity A has a response factor of 1.53 relative to anamorelin hydrochloride during high performance liquid chromatography.

46) The tablet according to claim 45, wherein impurity A has a response factor of 1.53 relative to anamorelin hydrochloride during high performance liquid chromatography as described in Example 3.

47) A tablet comprising anamorelin hydrochloride as an active ingredient and pharmaceutically acceptable carrier means for preventing the formation of impurity A.

48) The tablet of claim 41, wherein said carrier means comprises a pharmaceutically acceptable carrier selected from one or a combination of microcrystalline cellulose, croscarmellose sodium, silicon dioxide, magnesium stearate, anhydrous dibasic calcium phosphate, lactose monohydrate, D-mannitol, corn starch, low-substituted hydroxypropylcellulose, sodium starch glycolate, carmellose calcium, carmellose, crospovidone, partially pregelatinized maize starch, stearic acid and sodium stearyl fumarate.

49) The tablet according to claim 47, wherein said pharmaceutically acceptable carrier means act as HCl sequestrants in an intimate admixture with said anamorelin hydrochloride and compressed with said anamorelin hydrochloride at a compression force of from about 0.5 to about 15 kN.

50) The tablet according to any of claims 47 to 49, wherein said pharmaceutically acceptable carrier means act as HCl sequestrants in an intimate admixture with said anamorelin hydrochloride and compressed to a hardness of from about 40 to about 200 Newtons.

51) The tablet according to claim 47, wherein said pharmaceutically acceptable carrier means comprises one or a combination of carriers selected from microcrystalline cellulose, croscarmellose sodium, silicon dioxide, magnesium stearate, anhydrous dibasic calcium phosphate, lactose monohydrate, D-mannitol, corn starch, low-substituted hydroxypropylcellulose, sodium starch glycolate, carmellose calcium, carmellose, crospovidone, partially pregelatinized maize starch, stearic acid and sodium stearyl fumarate in an intimate admixture with said anamorelin hydrochloride and compressed with said anamorelin hydrochloride at a compression force of from about 0.5 to about 15 kN.

52) The tablet according to claim 47 or 51, wherein said pharmaceutically acceptable carrier means comprises one or a combination of carriers selected from microcrystalline cellulose, croscarmellose sodium, silicon dioxide, magnesium stearate, anhydrous dibasic calcium phosphate, lactose monohydrate, D-mannitol, corn starch, low-substituted hydroxypropylcellulose, sodium starch glycolate, carmellose calcium, carmellose, crospovidone, partially pregelatinized maize starch, stearic acid and sodium stearyl fumarate in an intimate admixture with said anamorelin hydrochloride and compressed to a hardness of from about 40 to about 200 Newtons.

53) The tablet according to any one of claims 47 to 52, comprising from about 0.01 to about 20 parts by weight of said pharmaceutically acceptable carrier means based on 1 part by weight of anamorelin hydrochloride.

54) The tablet according to claim 53, comprising from about 0.5 to about 10 parts by weight of said pharmaceutically acceptable carrier means based on 1 part by weight of anamorelin hydrochloride.

55) The tablet according to 54, comprising from about 1 to about 6 parts by weight of said pharmaceutically acceptable carrier means based on 1 part by weight of anamorelin hydrochloride.

56) The tablet according to any one of claims 47 to 55, wherein impurity A is not substantially produced or the amount of impurity A produced after storage for 2 to 6 months at 40° C. and a relative humidity of 75% is less than about 0.1% based on the weight of said anamorelin hydrochloride.

57) The tablet according to claim 56, wherein impurity A is not substantially produced or the amount of impurity A produced after storage for 2 months at 40° C. and a relative humidity of 75% is less than about 0.05% based on the weight of said anamorelin hydrochloride.

58) The tablet according to any one of claims 47 to 57, wherein impurity A has a response factor of 1.53 relative to anamorelin hydrochloride during high performance liquid chromatography.

59) The tablet according to any claim 58, wherein impurity A has a response factor of 1.53 relative to anamorelin hydrochloride during high performance liquid chromatography as described in Example 3.

60) A method for improving one or more symptoms of cancer cachexia in a patient in need thereof comprising administering to said patient a therapeutically effective amount of anamorelin hydrochloride in a tablet according to any of claims 35 to 59, wherein:
a) said patient is characterized by a body mass index less than 25, a score on the Cancer Fatigue Scale of from 20 to 28, or a Quality-of-Life Questionnaire for Cancer Patients Treated With Anticancer Drugs (QOL-ACD) score of from 65 to 80; and
b) said symptoms are selected from the group consisting of lean body mass, appetite, body weight, fatigue, and quality of life.

61) The method of claim 60, wherein the lean body mass is estimated by dual-energy x-ray absorptiometry (DEXA), the fatigue is measured by the Cancer Fatigue Scale, and the quality of life is measured by a QOL-ACD score for items 7 to 11 (“physical condition”), item 8 (“Did you have a good appetite?”), item 9 (“Did you enjoy your meals?”), and item 11 (“Did you lose any weight?”).

62) The method of claims 60 or 61, wherein the patient has stage III or IV non-small cell lung cancer (NSCLC) or advanced gastrointestinal (colorectal, gastric, or pancreatic) cancer.
